(19) United States
(12) Patent Application Publication
(10) Pub. No.: US 2013/0110005 A1
(43) Pub. Date: May 2, 2013

(54) POINT SIZE LIGHT ILLUMINATION IN METROLOGY SYSTEMS FOR IN-SITU SURGICAL APPLICATIONS

(71) Applicant: Covidien LP, Mansfield, MA (US)

(72) Inventor: Alexey Sharonov, Bethany, CT (US)

(73) Assignee: COVIDIEN LP, Mansfield, MA (US)

(21) Appl. No.: 13/645,559

(22) Filed: Oct. 5, 2012

Related U.S. Application Data
(60) Provisional application No. 61/551,961, filed on Oct. 27, 2011.

(51) Int. Cl. A61B 5/107

(52) U.S. Cl. 600/587

(57) ABSTRACT

A metrology method includes the steps of positioning a point source projector at a known distance from a target site, projecting a light beam through a semi-transparent mask, forming a magnified pattern on the target site from the light beam, visually inspecting a portion of the magnified pattern that is formed on the target site, and determining a measurement of the target site based on the known dimensions of the mask pattern, a magnification factor, and the portion of the magnified pattern that is formed on the target site. The light beam diverges from a point and the semi-transparent mask has a mask pattern of known dimensions. The magnified pattern is magnified from the mask pattern by the magnification factor.

### CROSS REFERENCE TO RELATED APPLICATION

The present application claims the benefit of and priority to U.S. Provisional Application Ser. No. 61/551,961, filed on Oct. 27, 2011, the entire contents of which are incorporated herein by reference.

### BACKGROUND

1. Technical Field

The present disclosure relates to a method for measuring a dimension of a target site. More particularly, the present disclosure relates to a method of projecting an image for use in measuring a dimension of a target site.

2. Background of the Related Art

Minimally invasive surgery, e.g., laparoscopic, endoscopic, and thoroscopic surgery, has many advantages over traditional open surgeries. In particular, minimally invasive surgery eliminates the need for a large incision, thereby reducing discomfort, recovery time, and many of the deleterious side effects associated with traditional open surgery.

The minimally invasive surgeries are performed through small openings in a patient's skin. These openings may be incisions in the skin or may be naturally occurring body orifices (e.g., mouth, anus, or vagina). In general, insufflation gas is used to enlarge the area surrounding the target surgical site to create a larger, more accessible work area.

During minimally invasive procedures, it is often difficult for a surgeon to determine sizes of various organs, tissues, and other structures in a surgical site. Various in-situ surgical metrology methods exist for measurement in a surgical site. Such methods require many moving parts and projection images that change size and/or focus quickly as projectors move in or out of a surface of projection. A continuing need exists for in-situ surgical metrology methods that operate with a stable focus and no moving parts.

### SUMMARY

A metrology method includes the steps of positioning a point source projector at a known distance from a target site, projecting a light beam through a semi-transparent mask, forming a magnified pattern on the target site from the light beam, visually inspecting a portion of the magnified pattern that is formed on the target site, and determining a measurement of the target site based on the known dimensions of the mask pattern, a magnification factor, and the portion of the magnified pattern that is formed on the target site. The light beam diverges from a point and the semi-transparent mask has a mask pattern of known dimensions. The magnified pattern is magnified from the mask pattern by the magnification factor.

The light beam may be a laser emitted by a laser diode disposed within the point source projector. The light beam may be emitted by an LED disposed within the point source projector. The light beam may be focused to the point by a lens disposed within the point source projector. The semi-transparent mask may be translatable to adjust the magnification factor. The semi-transparent mask may be disposed within the point source projector. The point source projector may be attached to an endoscope for visually inspecting the target site. The mask pattern may include a series of uniformly spaced concentric circles. The mask pattern may also or alternatively include a series of uniformly spaced linear markings.

### BRIEF DESCRIPTION OF THE DRAWINGS

The above and other aspects, features, and advantages of the present disclosure will become more apparent in light of the following detailed description when taken in conjunction with the accompanying drawings in which:

FIG. 1 is a side, schematic view of a metrology system according to the principles of the present disclosure;

FIG. 2 is a side, perspective view of a method of use of the metrology system of FIG. 1; and

FIG. 3 is a side, schematic view of a metrology system according to another embodiment of the present disclosure.

### DETAILED DESCRIPTION

Particular embodiments of the present disclosure are described hereinbelow with reference to the accompanying drawings; however, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

Like reference numerals may refer to similar or identical elements throughout the description of the figures. As shown in the drawings and described throughout the following description, as is traditional when referring to relative positioning on a surgical instrument, the term “proximal” refers to the end of the apparatus which is closer to the user and the term “distal” refers to the end of the apparatus which is farther away from the user. The term “clinician” refers to any medical professional (i.e., doctor, surgeon, nurse, or the like) performing a medical procedure involving the use of embodiments described herein.

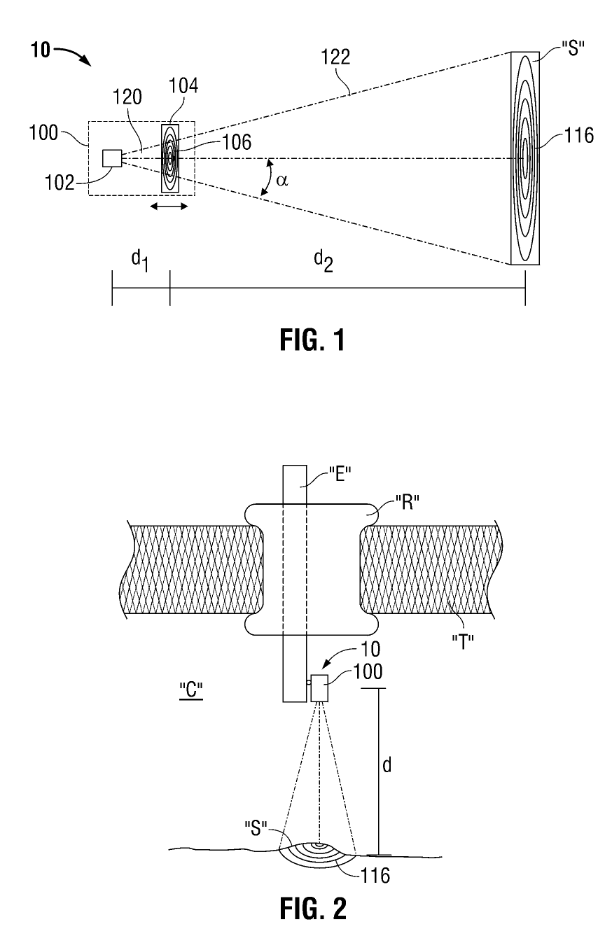

As seen in FIG. 1, a metrology system 10 includes a point source projector 100 having a point source light emitter 102 and a mask 104. Mask 104 is a distance d₁ from point source light emitter 102 and a distance d₂ from a target site “S”. Mask 104 is semi-transparent and has a substantially opaque mask pattern 106 thereon. Mask pattern 106 has markings of known distances therebetween. For example, mask pattern 106 may be a series of uniformly spaced concentric circles. Mask 104 may be translatable toward or away from point source light emitter 102.

Point source light emitter 102 emits a light beam 120 therefrom. Light beam 120 approximates a point at point source light emitter 102 and conically diverges therefrom at an angle α. Point source light emitter 102 may be any device capable of emitting light from a narrow point, such as a laser diode or an LED. Light beam 120 is partially blocked by mask pattern 106 upon incidence with mask 104. An unblocked portion 122 of light beam 120 continues past mask 104 to reach target site “S”. Unblocked portion 122 creates a magnified pattern 116 on target site “S”. Magnified pattern 116 is magnified from mask pattern 106 according to a formula: M=1+d₂/d₁, where M is a magnification factor between mask pattern 106 and magnified pattern 116. A translation of mask 104 or point source projector 100 away from target site “S” increases magnification factor M. A translation of mask 104 or point source projector 100 toward target site “S” decreases magnification factor M. Magnified pattern 116 retains a substantially sharp focus as mask 104 and/or point source projector 100 is translated.

A method of use of metrology system 10 is depicted in FIG. 2. Metrology system 10 is attached to a distal end of an endoscope “E”. Endoscope “E” is inserted into a body cavity “C” through an opening in a tissue “T”. Endoscope “E” may be inserted through a seal anchor “R” positioned within the opening in tissue “T”. Endoscope “E” is inserted through a port in seal anchor “R” that is expanded to a width greater than a maximum combined width of endoscope “E” and point source projector 100. Once the distal end of endoscope “E” is distal to seal anchor “R”, the port resiliently compresses to form a substantially airtight seal around endoscope “E”. Point source projector 100 is translated distally toward target site “S” until point source projector 100 arrives at a known distance d from target site “S”. The arrival of point source projector 100 at distance d may be determined through any appropriate means, such as triangulation. Distance d₁ may be fixed prior to insertion of endoscope “E”. Alternatively, endoscope “E” may include a mechanism, such as a rotatable knob (not shown), for altering distance d₁. Distance d₂ is calculated by subtracting distance d₁ from distance d. Distance d₁ and distance d₂ may then be used to calculate magnification factor M.

Point source projector 100 projects magnified pattern 116 onto target site “S”. A clinician may observe magnified pattern 116 through endoscope “E”. A dimension of target site “S” is measured by visually inspecting and counting a number n of uniformly spaced markings appearing along the dimension of target site “S”. The number n of uniformly spaced markings is multiplied by a uniform distance between individual markings of pattern 116. The uniform distance between individual markings of pattern 116 is calculated by multiplying a uniform distance dₖ between individual markings of mask 104 by magnification factor M. Thus, a measure of the dimension of target site “S” is calculated according to the formula: x=nMdₖ, where x is the measure of the dimension.

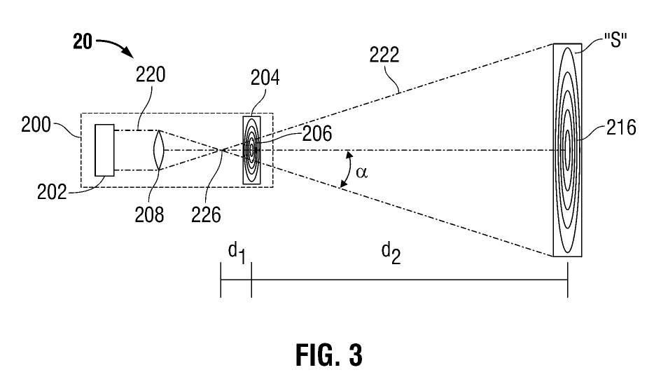

Turning to FIG. 3, a metrology system in accordance with an alternate embodiment of the present disclosure is generally designated as 20. Metrology system 20 is similar to metrology system 10 and thus will only be discussed as necessary to identify the differences in construction and operation thereof.

Metrology system 20 includes a point source projector 200 having a light source 202, a mask 204, and a lens 208. Mask 204 has a mask pattern 206. Light source 202 emits a light beam 220 toward lens 208. Lens 208 is a converging lens that focuses light beam 220 into a point 226. Point 226 is a distance d₁ away from mask 204. Light beam 220 diverges at an angle α from point 226 and is partially blocked by mask 204. An unblocked beam 222 passes through mask 204 and travels a distance d₂ to a target site “S” to form a magnified pattern 216 thereon.

A method of use of metrology system 20 is substantially identical to the method of use of metrology system 10 described hereinabove.

It should be understood that the foregoing description is only illustrative of the present disclosure. Various alternatives and modifications can be devised by those skilled in the art without departing from the disclosure. Accordingly, the present disclosure is intended to embrace all such alternatives, modifications and variances. The embodiments described with reference to the attached drawing figs. are presented only to demonstrate certain examples of the disclosure. Other elements, steps, methods and techniques that are insubstantially different from those described above and/or in the appended claims are also intended to be within the scope of the disclosure.

What is claimed is:

1. A metrology method, comprising the steps of:
positioning a point source projector at a known distance from a target site;
projecting a light beam through a semi-transparent mask, wherein the light beam diverges from a point and the semi-transparent mask has a mask pattern of known dimensions;
forming a magnified pattern on the target site from the light beam, wherein the magnified pattern is magnified from the mask pattern by a magnification factor;
visually inspecting a portion of the magnified pattern that is formed on the target site; and
determining a measurement of the target site based on the known dimensions of the mask pattern, the magnification factor, and the portion of the magnified pattern that is formed on the target site.

2. A metrology method according to claim 1, wherein the light beam is a laser emitted by a laser diode disposed within the point source projector.

3. A metrology method according to claim 1, wherein the light beam is emitted by an LED disposed within the point source projector.

4. A metrology method according to claim 1, wherein the light beam is focused to the point by a lens disposed within the point source projector.

5. A metrology method according to claim 1, wherein the semi-transparent mask is translatable to adjust the magnification factor.

6. A metrology method as in claim 1, wherein the semi-transparent mask is disposed within the point source projector.

7. A metrology method according to claim 1, wherein the point source projector is attached to an endoscope for visually inspecting the target site.

8. A metrology method according to claim 1, wherein the mask pattern includes a series of uniformly spaced concentric circles.

9. A metrology method according to claim 1, wherein the mask pattern includes a series of uniformly spaced linear markings.
